(19) United States
(12) Patent Application Publication
(10) Pub. No.: US 2023/0172658 A1
(43) Pub. Date: Jun. 8, 2023

(54) CATHETER AND PERFORATION SYSTEM INCLUDING CATHETER

(71) Applicant: Boston Scientific Medical Device Limited, Galway (IE)

(72) Inventors: Maria Luk, Kleinburg (CA); Gareth Davies, Toronto (CA); John Paul Urbanski, Toronto (CA)

(21) Appl. No.: 17/917,636

(22) PCT Filed: Apr. 6, 2021
(86) PCT No.: PCT/IB2021/052851

§ 371 Date: Oct. 7, 2022

Related U.S. Application Data
(60) Provisional application No. 63/007,662, filed on Apr. 9, 2020.

(51) Int. Cl. A61B 18/14; A61L 29/08; A61L 29/12; A61L 29/14

(52) U.S. Cl. CPC A61B 18/1492; A61L 29/085; A61L 29/126; A61L 29/14; A61B 2090/3966

(57) ABSTRACT

A catheter includes an elongate shaft extending longitudinally between a proximal portion defining a proximal end and a distal portion defining a distal end. A lumen extends through the shaft from the proximal end to the distal end. The shaft includes a heat-shielding layer and an outer layer adjacent the heat-shielding layer. The heat-shielding layer includes an inner liner layer adjacent the lumen and a heat-shielding layer adjacent the inner liner layer. In the distal portion, at least a section of the outer layer is radiopaque.

### FIELD

This document relates to catheters. More specifically, this document relates to catheters usable in medical procedures, and to perforation systems including catheters.

### SUMMARY

The following summary is intended to introduce the reader to various aspects of the detailed description, but not to define or delimit any invention.

Catheters are disclosed. According to some aspects, a catheter includes an elongate shaft extending longitudinally between a proximal portion defining a proximal end and a distal portion defining a distal end. A lumen extends through the shaft from the proximal end to the distal end. The shaft includes a heat-shielding layer and an outer layer adjacent the heat-shielding layer. The heat-shielding layer includes an inner liner layer adjacent the lumen, an intermediate layer adjacent the inner liner layer. In the distal portion, at least a section of the outer layer is radiopaque.

In some examples, the liner layer includes a first polymer. The first polymer can be or can include polytetrafluoroethylene (PTFE).

In some examples, the intermediate layer includes a second polymer. The second polymer can be melt processable and flexible. The second polymer can be or can include at least one of a polyether block amide (PEBA), an aliphatic polyether-based thermoplastic polyurethane (TPU), a nylon, a polyurethane, and a polyethylene. In some examples, the second polymer is polyether block amide.

In some examples, the outer layer includes a third polymer. The third polymer can be or can include at least one of a polyether block amide (PEBA), an aliphatic polyether-based thermoplastic polyurethane (TPU), a nylon, a polyurethane, and a polyethylene. In some examples, the third polymer is polyether block amide (PEBA).

In some examples, in the section that is radiopaque, the third polymer is filled with a radiopaque filler. The radiopaque filler can be or can include at least one of tungsten, barium sulphate, and bismuth. In some examples, the radiopaque filler includes tungsten.

In some examples, the section that is radiopaque extends along an entirety of the distal portion.

In some examples, the section that is radiopaque includes a radiopaque band in the distal portion. The radiopaque band can be at the distal end.

In some examples, the distal portion has a length of between about 1 mm and about 5 mm.

In some examples, the liner layer, intermediate layer, and outer layer are of a constant thickness between the proximal end and the distal end.

In some examples, the proximal portion includes at least a first tapered section adjacent the distal portion, and the outer layer tapers in thickness in the tapered section.

In some examples, the distal portion is tapered.

Perforation systems are also disclosed. According to some aspects, a perforation system includes a catheter, a perforation device, and a radiofrequency generator. The catheter includes an elongate shaft extending longitudinally between a proximal portion defining a proximal end and a distal portion defining a distal end, and a lumen extending through the shaft from the proximal end to the distal end. The shaft includes a heat-shielding layer and an outer layer adjacent the heat-shielding layer. The heat-shielding layer includes an inner liner layer adjacent the lumen, and an intermediate layer adjacent the inner liner layer. In the distal portion, at least a section of the outer layer is radiopaque. The perforation device includes a shaft receivable in the catheter and having a heat-generating radiofrequency electrode positionable proximate the distal end of the catheter. The radiofrequency generator is connectable to the perforation device to supply radiofrequency energy to the radiofrequency electrode.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example perforation system;

FIG. 2 is a side view of the catheter of the system of FIG. 1, showing a lumen in dotted line;

FIG. 3 is an enlarged partial side view of the catheter of FIG. 2;

FIG. 4 is a cross-section taken along line 4-4 in FIG. 3;

FIG. 5 is a partial side view of another example catheter;

FIG. 6 is a cross-section taken along line 6-6 in FIG. 5;

FIG. 7 is a partial side view of another example catheter; and

FIG. 8 is a cross-section taken along line 8-8 in FIG. 7.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are catheters and related systems. The catheters can be used in various medical procedures, such as (but not limited to) perforation procedures. As will be described in further detail below, the catheters disclosed herein have at least one radiopaque section in the distal portion thereof. For example, the entire length of the distal portion can be radiopaque, or the distal portion can include one or more radiopaque bands. During a medical procedure, the radiopaque section can be viewed under fluoroscopy, to facilitate proper positioning of the catheter. Furthermore, as will be described below, the distal portion is configured so that the radiopaque section is shielded from heat (e.g. heat generated by other medical devices). This can allow for the radiopaque section to be positioned at or proximate the distal end of the catheter, while preventing or inhibiting failure of the radiopaque section due to heat exposure.

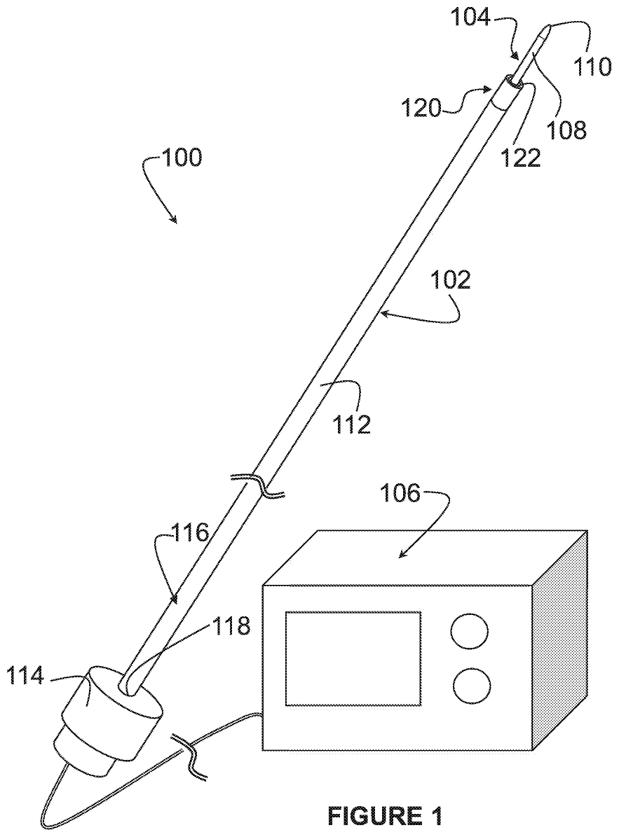

Referring now to FIG. 1, an example system 100 is shown. The system 100 is a transseptal perforation system, for advancing towards a patient's heart and perforating a fossa ovalis of the patient's heart.

In the example shown, the system 100 includes a catheter 102, a radiofrequency (RF) perforation device 104, and an RF generator 106. The RF perforation device 104 includes an elongate body 108 and an RF electrode 110. The RF perforation device 104 is connectable to the RF generator 106, so that the RF generator 106 can supply RF energy to the RF electrode 110. Optionally, the system 100 can further include a dilator (not shown).

In one example of use, the catheter 102 can be advanced intravenously via the femoral vein towards the right atrium of the patient's heart. The RF perforation device 104 can be connected to the RF generator 106, which can in turn be connected to one or more grounding pads (not shown). The RF perforation device 104 can then be advanced through the catheter 102 (optionally via the dilator). When the catheter 102 is in the desired position in the patient's heart, for example adjacent the fossa ovalis, the RF perforation device 104 can be advanced to position the RF electrode 110 adjacent the fossa ovalis, and the RF generator 106 can be activated to deliver RF energy to the RF electrode 110, to perforate the fossa ovalis. Such procedures can be carried out, for example, as a medical treatment, or to gain access to the left atrium for a subsequent medical treatment.

As mentioned above and as will be described in further detail below, the catheter 102 can include at least one radiopaque section in the distal portion thereof. During the procedure, fluoroscopy can be used to visualize the radiopaque section and thereby confirm or determine or check the position of the catheter 102 within the patient's body. However, delivery of RF energy can cause significant heat to be generated by the RF electrode 110 (i.e. the RF electrode is heat generating), and heat can cause failure of radiopaque materials. The catheter 102 is thus configured such that the radiopaque section is shielded from heat generated by the RF electrode 110.

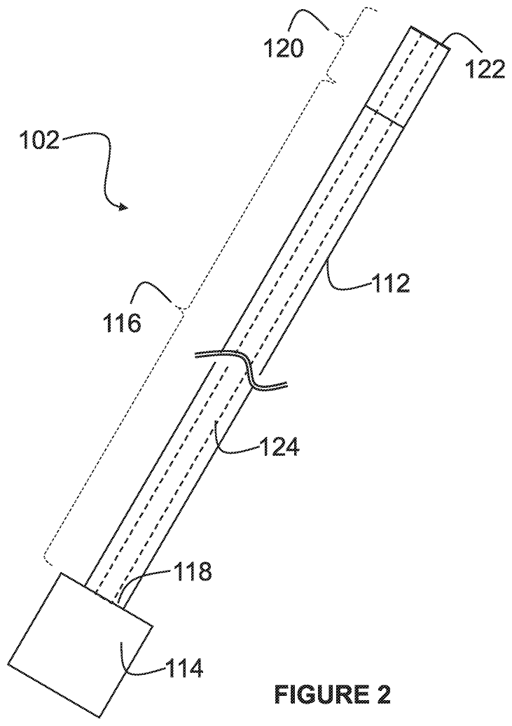

Referring now to FIG. 2, the catheter 102 is shown in greater detail. In the example shown, the catheter 102 includes an elongate shaft 112 and a handle 114. The shaft 112 extends longitudinally between a proximal portion 116 defining a proximal end 118 and a distal portion 120 defining a distal end 122. The handle 114 is mounted to the proximal end 118.

In the example shown, the proximal portion 116 makes up a majority of the length of the shaft 112, while the distal portion 120 makes up a relatively small portion of the length. For example, the proximal portion 116 can be sufficiently long to extend between the femoral vein and the heart (e.g. approximately 250 cm), while the distal portion 120 can be only a few centimeters (e.g. between 1 cm and 5 cm, or about 3 cm).

Referring still to FIG. 2, in the example shown, the catheter 102 includes a lumen 124 (shown in dotted line) that extends through the shaft 112, from the proximal end 118 to the distal end 122. The lumen 124 can accommodate various other medical devices, such as the RF perforation device 104 and a dilator.

As will be described below, the shaft 112 includes a plurality of layers, at least one of which includes a radiopaque section, and at least another of which is configured as a heat-shielding layer to shield the radiopaque section from heat.

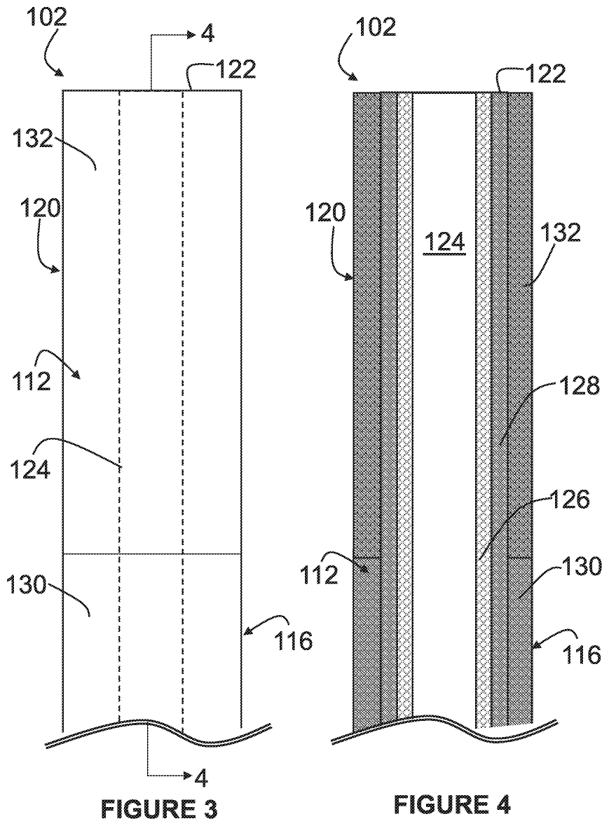

Referring to FIGS. 3 and 4, in the example shown, the shaft 112 includes a heat-shielding layer that is made up of two sub-layers: an inner liner layer 126 and an intermediate layer 128. The inner liner layer 126 is adjacent the lumen 124 and defines the lumen 124. In the example shown, the liner layer 126 extends from the proximal end 118 (not shown in FIGS. 3 and 4) to the distal end 122; in alternative examples, the liner layer can extend along only a portion of the shaft (e.g. the liner layer can extend along only the distal portion, and the proximal portion can be of a different configuration). The liner layer 126 can be or can include a polymer (referred to herein as a ‘first polymer’). The first polymer can be any suitable polymer that, when layered with the intermediate layer 128, can shield can shield additional layers from heat emanating from a medical device received within the catheter 102. For example, the first polymer can be polytetrafluoroethylene (PTFE).

Referring still to FIGS. 3 and 4, the intermediate layer 128 is adjacent the liner layer 126. In the example shown, the intermediate layer 128 extends from the proximal end 118 (not shown in FIGS. 3 and 4) to the distal end 122; in alternative examples, the intermediate layer can extend along only a portion of the shaft (e.g. the intermediate layer can extend along only the distal portion, and the proximal portion can be of a different configuration). The intermediate layer 128 can be or can include a polymer (referred to herein as a ‘second polymer’, which can be the same or different from the first polymer). The second polymer can be any suitable polymer that, when layered with the first polymer, can shield additional layers from heat emanating from a medical device received within the catheter 102. Furthermore, the second polymer can be flexible, to facilitate maneuvering of the catheter 102, and can be melt-processable, to facilitate production of the catheter 102 by melt-flowing the second polymer onto the liner layer 126. In some examples, the second polymer can be or can include a polyether block amide (PEBA) (e.g. a polymer sold under the brand name PEBAX®), an aliphatic polyether-based thermoplastic polyurethane (TPU) (e.g. a polymer sold under the brand tam Tecoflex®), a nylon, a polyurethane, and/or a polyethylene. In one particular example, the second polymer is PEBA.

Referring still to FIGS. 3 and 4, the shaft 112 further includes an outer layer 130 adjacent the intermediate layer 128. In the example shown, the outer layer 130 extends from the proximal end 118 (not shown in FIGS. 3 and 4) to the distal end 122; in alternative examples, the outer layer can extend along only a portion of the shaft (e.g. the outer layer can extend along only the distal portion, and the proximal portion can be of a different configuration).

In general, in the distal portion 120, at least a section (also referred to herein as a ‘radiopaque section’) of the outer layer 130 is radiopaque. Referring to FIGS. 3 and 4, in the example shown, the radiopaque section 132 extends along an entirety of the distal portion 120; however in alternative examples, the outer layer can include a relatively small radiopaque band in the distal portion, or several spaced apart radiopaque bands (as described below).

Referring still to FIGS. 3 and 4, the outer layer 130 can be or can include a third polymer (which can be the same as or different from the first and second polymers), and in the radiopaque section 132, the third polymer can be filled with a radiopaque filler. The third polymer can be flexible, to facilitate maneuvering of the catheter 102, and can be melt-processable, to facilitate production of the catheter 102 by melt-flowing the third polymer onto the intermediate layer 128. For example, the third polymer can be or can include a PEBA, a TPU, a nylon, a polyurethane, and a polyethylene. In one particular example the third polymer and the second polymer are both PEBA. Furthermore, in the radiopaque section 132, the third polymer can be filled with a radiopaque filler such as tungsten, barium sulphate, and/or bismuth. In one particular example, the radiopaque section includes PEBA filled with 80 wt % tungsten.

In the example shown, the liner layer 126, intermediate layer 128, and outer layer 130 are of a constant thickness between the proximal end 118 and the distal end 122. In alternative examples, as will be described below, one or more of the layers may have a varying thickness. For example, one or more of the layers may be tapered.

In the example shown, the liner layer 126, intermediate layer 128, and outer layer 130 all extend to the distal end 122 of the shaft. In alternative examples, one or more of these layers may extend to a position that is slightly shy of the distal end of the shaft. For example, the inner liner layer may extend to and define the distal end of the shaft. The intermediate layer and outer layer may extend to a position that is slightly shy of the distal end of the shaft, i.e. so that the inner liner layer extends proud of the intermediate layer and outer layer. This can further shield the radiopaque section of the outer layer from heat.

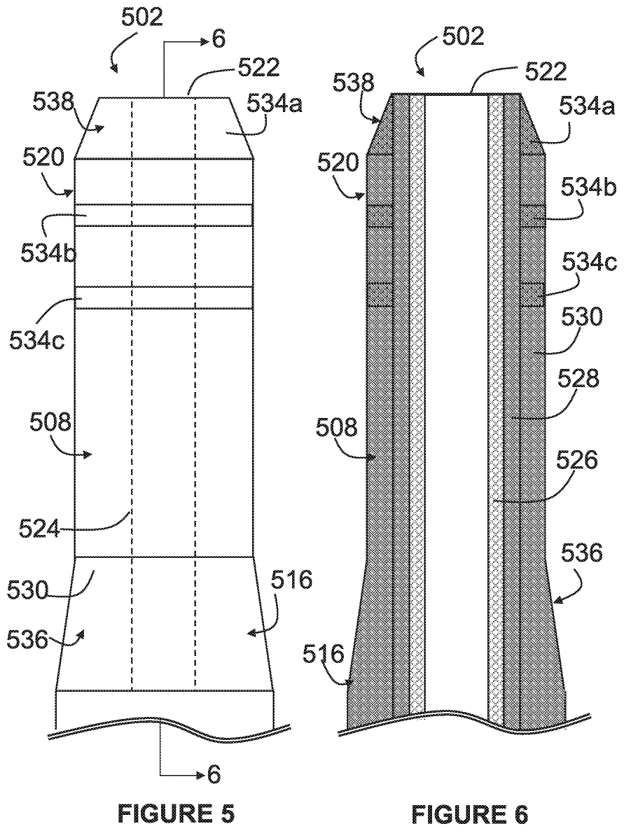

Referring now to FIGS. 5 and 6, an alternative example of a catheter is shown. In FIG. 5, features that are similar to those of FIGS. 1 to 4 are referenced with like reference numerals, incremented by 400.

Similarly to the catheter 102 of FIGS. 1 to 4, the catheter 502 of FIGS. 5 and 6 includes an elongate shaft 508 that has a proximal portion 516 defining a proximal end (not shown) and a distal portion 520 defining a distal end 522. The shaft 508 has a liner layer 526 and an intermediate layer 528 that form a heat-shielding layer, and an outer layer 530. The catheter 502 further includes a lumen 524.

Referring still to FIGS. 5 and 6, in the distal portion 520, the outer layer 530 includes a set of radiopaque bands 534a-534c. Similarly to the radiopaque section 132 of FIGS. 1 to 4, the radiopaque bands 534a-534c can be formed by filling the polymer of the outer layer 530 with a radiopaque filler. In the example shown, the outer layer 530 includes a first radiopaque band 534a that is at the distal end 522, and second 534b and third 534c radiopaque bands spaced proximally from the first radiopaque band 534a.

Referring still to FIGS. 5 and 6, in the example shown, the shaft 508 includes two tapered sections, to provide the distal end 522 with a smaller profile than the proximal end. Particularly, the proximal portion 516 includes a first tapered section 536 adjacent the distal portion 520. The outer layer 530 tapers in thickness in the first tapered section 536. Furthermore, the distal portion 520 includes a second tapered section 538, at the distal end 522. The outer layer 530 tapers in thickness again in the second tapered section 538. The second tapered section 538 includes the first radiopaque band 534a.

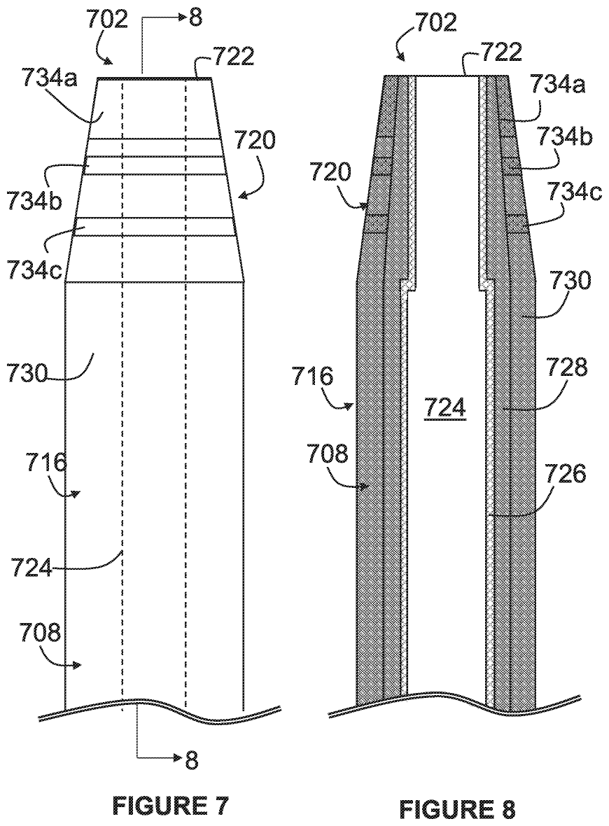

Referring now to FIGS. 7 and 8, an alternative example of a catheter is shown. In FIGS. 7 and 8, features that are similar to those of FIGS. 5 and 6 are referenced with like reference numerals, incremented by 200.

Similarly to the catheter of FIGS. 5 and 6, the catheter 702 of FIGS. 7 and 8 includes an elongate shaft 708 that has a proximal portion 716 defining a proximal end (not shown) and a distal portion 720 defining a distal end 722. The shaft 708 has a liner layer 726 and an intermediate layer 728 that form a heat-shielding layer, and an outer layer 730. The catheter 702 further includes a lumen 724.

Referring still to FIGS. 7 and 8, in the distal portion 720, the outer layer 730 includes a set of radiopaque bands 734a-734c. Similarly to the radiopaque section 132 of FIGS. 1 to 4, the radiopaque bands 734a-734c can be formed by filling the polymer of the outer layer 730 with a radiopaque filler. In the example shown, the outer layer 730 includes a first radiopaque band 734a that is at the distal end 722, and second 734b and third 734c radiopaque bands spaced proximally from the first radiopaque band 734a.

Referring still to FIGS. 7 and 8, in the example shown, the entire distal portion 720 is tapered, to provide the distal end 722 with a smaller profile than the proximal end. Particularly, in the distal portion 720, the liner layer 726 is stepped, to provide a smaller cross-sectional area at the distal end 722. Furthermore, the intermediate layer 728 tapers in diameter area going towards the distal end 722. Finally, the outer layer 730, including the radiopaque bands 734a-734c, tapers in thickness going towards the distal end 722.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A catheter comprising:
an elongate shaft extending longitudinally between a proximal portion defining a proximal end and a distal portion defining a distal end; and
a lumen extending through the shaft from the proximal end to the distal end;
wherein the shaft comprises a heat-shielding layer and an outer layer adjacent the heat-shielding layer;
wherein the heat-shielding layer comprises an inner liner layer adjacent the lumen, and an intermediate layer adjacent the inner liner layer; and
wherein in the distal portion, at least a section of the outer layer is radiopaque.

2. The catheter of claim 1, wherein the liner layer comprises a first polymer.

3. The catheter of claim 2, wherein the first polymer comprises polytetrafluoroethylene (PTFE).

4. The catheter of claim 1, wherein the intermediate layer comprises a second polymer, wherein the second polymer is melt processable and flexible.

5. The catheter of claim 4, wherein the second polymer comprises at least one of a polyether block amide (PEBA), an aliphatic polyether-based thermoplastic polyurethane (TPU), a nylon, a polyurethane, and a polyethylene.

6. The catheter of claim 5, wherein the second polymer comprises polyether block amide.

7. The catheter of claim 1, wherein the outer layer comprises a third polymer.

8. The catheter of claim 7, wherein the third polymer comprises at least one of a polyether block amide (PEBA), an aliphatic polyether-based thermoplastic polyurethane (TPU), a nylon, a polyurethane, and a polyethylene.

9. The catheter of claim 7, wherein the third polymer comprises polyether block amide (PEBA).

10. The catheter of claim 1, wherein in the section that is radiopaque, the third polymer is filled with a radiopaque filler.

11. The catheter of claim 10, wherein the radiopaque filler comprises at least one of tungsten, barium sulphate, and bismuth.

12. The catheter of claim 11, wherein the radiopaque filler comprises tungsten.

13. The catheter of claim 1, wherein the section that is radiopaque extends along an entirety of the distal portion.

14. The catheter of claim 8, wherein the section that is radiopaque comprises a radiopaque band in the distal portion.

15. The catheter of claim 14, wherein the radiopaque band is at the distal end.

16. The catheter of claim 1, wherein the distal portion has a length of between about 1 mm and about 5 mm.

17. The catheter of claim 1, wherein the liner layer, intermediate layer, and outer layer are of a constant thickness between the proximal end and the distal end.

18. The catheter of claim 1, wherein the proximal portion comprises at least a first tapered section adjacent the distal portion, and the outer layer tapers in thickness in the tapered section.

19. The catheter of claim 1, wherein the distal portion is tapered.

20. A perforation system comprising:
a catheter comprising i) an elongate shaft extending longitudinally between a proximal portion defining a proximal end and a distal portion defining a distal end, and ii) a lumen extending through the shaft from the proximal end to the distal end, wherein the shaft comprises a heat-shielding layer and an outer layer adjacent the heat-shielding layer, wherein the heat-shielding layer comprises an inner liner layer adjacent the lumen and an intermediate layer adjacent the inner liner layer, and wherein in the distal portion, at least a section of the outer layer is radiopaque;
a perforation device comprising an elongate body receivable in the catheter and having a heat-generating radiofrequency electrode positionable proximate the distal end of the catheter; and
a radiofrequency generator connectable to the perforation device to supply radiofrequency energy to the radiofrequency electrode.
